(19) United States
(12) Patent Application Publication
(10) Pub. No.: US 2024/0324561 A1
(43) Pub. Date: Oct. 3, 2024

(54) SYSTEM FOR INSECT LARVAE HARVESTING

(71) Applicant: AgGen, Inc., Davis, CA (US)

(72) Inventors: Amir Salehzadeh, Davis, CA (US); Rosh Gabriel-Jia Cheng Ho, Davis, CA (US)

(21) Appl. No.: 18/126,952

(22) Filed: Mar. 27, 2023

(51) Int. Cl. A01K 67/033

(52) U.S. Cl. CPC A01K 67/033

(57) ABSTRACT

An electric insect migration harvesting apparatus includes a container, a first conductive element and a second conductive element positioned within the container, an extraction zone, an access configured to provide access to the extraction zone from outside of the container, a positive electric terminal and a negative electric terminal. The positive electric terminal is electrically connected to the first conductive element and the negative electric terminal is electrically connected to the second conductive element. The second conductive element is located closer to the extraction zone than the first conductive element. A positive terminal of the electric energy source is electrically connected to the positive electric terminal and a negative terminal of the electric energy source is electrically connected to the negative electric terminal. A switch may be configured to connect or disconnect a terminal of the electric energy source to the first conductive element or the second conductive element.

### TECHNICAL FIELD

The present invention generally relates to systems and methods harvesting insects from a substrate.

### BACKGROUND INFORMATION

Insects, such as black soldier fly larvae (BSFL), are bred and harvested for a variety of uses. One of these uses is animal feed. Insects, such as black soldier fly larvae, are a great source of protein and can be used as feed for livestock, poultry, and even fish. They are particularly useful for aquaculture as they have a high fat content that is beneficial for fish growth. Another use is waste management. Insect larvae are excellent at consuming organic waste, such as food scraps and manure. This makes them useful for composting and reducing waste in landfills. Another use is human food. Insect larvae are consumed as a traditional food in some parts of the world. They are a good source of protein and are often fried or roasted. Yet another use is for soil amendment. Insect larvae can be used as a soil amendment to improve soil fertility and structure. They break down organic matter and release nutrients that are beneficial for plant growth. Another use is for medical research. Insect larvae have been used in medical research to study wound healing, antibiotic resistance, and other health-related topics. Insect larvae have a wide range of uses and are becoming increasingly popular due to their ability to convert waste into valuable resources.

### SUMMARY

In a first novel aspect, an electric insect migration harvesting apparatus includes a container, a first conductive element and a second conductive element positioned within the container, an extraction zone, an access configured to provide access to the extraction zone from outside of the container, a positive electric terminal and a negative electric terminal. The positive electric terminal is electrically connected to the first conductive element and the negative electric terminal is electrically connected to the second conductive element. The second conductive element is located closer to the extraction zone than the first conductive element. A positive terminal of the electric energy source is electrically connected to the positive electric terminal and a negative terminal of the electric energy source is electrically connected to the negative electric terminal. A switch may be configured to connect or disconnect a terminal of the electric energy source to the first conductive element or the second conductive element.

In a second novel aspect, the electric insect migration harvesting apparatus also includes a switch configured to connect or disconnect a terminal of the electric energy source to the first conductive element or the second conductive element.

In a third novel aspect, the container is configured to house a biological substrate material.

In a fifth novel aspect, the first conductive element and the second conductive element are positioned within the container such that the majority of an interior volume of the container is between the first conductive element and the second conductive element.

In a sixth novel aspect, the first conductive element and the second conductive element are configured such that a current flows between the first conductive element and the second conductive element when a first electric potential is applied to the first conductive element and a second electric potential is applied to the second conductive element.

In a seventh novel aspect, the container is configured to rear an insect, such as a Black Soldier Fly Larvae (BSFL).

In an eighth novel aspect, the container is configured to be heated or cooled. Heating or cooling may be achieved by insertion of hot or cold air, hot or cold water, or by heating or cooling the environment surrounding the apparatus.

In a ninth novel aspect, the container is configured to allow entry of air from outside the container.

In a tenth novel aspect, the container is configured to allow entry of water from outside the container.

In an eleventh novel aspect, the apparatus includes a vacuum tube configured to apply a vacuum to the extraction area.

In a twelfth novel aspect, the vacuum tube comprises a substrate filter configured to block the flow of substrate while allowing the passage of a larvae.

In a thirteenth novel aspect, the access is configured to mate with a vacuum nozzle.

In a fourteenth novel aspect, the apparatus includes a lid configured to cover the access.

In a fifteenth novel aspect, a method includes the steps of (a) applying a first electrical potential to a first conductive element positioned at a first position in a biological substrate, (b) apply a second electrical potential to a second conductive element position at a second position in the biological substrate, (c) waiting a first duration of time, wherein an insect in the biological substrate moves in a direction of current flow between the first conductive element and the second conductive element, and (d) disconnecting the first electrical potential from the first conductive element or disconnecting the second electrical potential form the second conductive element.

In a sixteenth novel aspect, the method also includes (e) collecting the insect from an extraction zone located adjacent to the second conductive element.

In a seventeenth novel aspect, an apparatus includes a container, an extraction zone located within the container, a positive electric terminal, a negative electric terminal, and a first means for causing one or more insects in the container to move in a first direction.

In an eighteenth novel aspect, the first means includes a first conductive element and a second conductive element. The positive electric terminal is electrically connected to the first conductive element, and the negative electric terminal is electrically connected to the second conductive element.

In a nineteenth novel aspect, the apparatus includes a second means for extracting the one or more insects from the extraction zone.

In a twentieth novel aspect, the second means includes an opening that provides access to the interior of container.

Further details and embodiments and techniques are described in the detailed description below. This summary does not purport to define the invention. The invention is defined by the claims.

### BRIEF DESCRIPTION OF THE DRAWINGS

The accompanying drawings, where like numerals indicate like components, illustrate embodiments of the invention.

FIG. 1 is a first diagram of a container housing insects being reared in a biological substrate.

FIG. 2 is a diagram of manual insect harvesting utilizing a sifter.

FIG. 3 is a first simplified diagram of an electric insect migration control system.

FIG. 4 is a second simplified diagram of an electric insect migration control system connected to a power source.

FIG. 5 is a third simplified diagram of an electric insect migration control system connected to a power source after the insect migration is completed.

FIG. 6 is a perspective view diagram of an insect rearing container.

FIG. 7 is a perspective view diagram of the internal parts of an electric insect migration harvesting apparatus.

FIG. 8 is a top-down view diagram of the internal parts of an electric insect migration harvesting apparatus.

FIG. 9 is a top-down view diagram of the internal parts of an electric insect migration harvesting apparatus housing a plurality of insect larvae.

FIG. 10 is a top-down view diagram of the internal parts of an electric insect migration harvesting apparatus after a plurality of insect larvae have migrated towards the second conductive element.

FIG. 11 is a perspective view diagram of the internal parts of an electric insect migration harvesting apparatus after insect migration has been completed.

FIG. 12 is a perspective view diagram of an insect rearing container including an optional vacuum nozzle.

FIG. 13 is a flowchart diagram illustrating the steps of electric insect migration harvesting.

### DETAILED DESCRIPTION

Reference will now be made in detail to background examples and some embodiments of the invention, examples of which are illustrated in the accompanying drawings. In the description and claims below, relational terms such as “top”, “down”, “upper”, “lower”, “top”, “bottom”, “left” and “right” may be used to describe relative orientations between different parts of a structure being described, and it is to be understood that the overall structure being described can actually be oriented in any way in three-dimensional space.

Insect larvae are typically reared in controlled environments, such as a rearing facility or a container, where they are provided with suitable food and conditions for growth. Once the rearing process has completed and the larvae have reached the desired stage of development, they are separated from their rearing substrate, which can be done manually or mechanically. The separation can involve sifting, sieving, or using air flow to separate the larvae from the substrate. After separation the larvae may be cleaned. The cleaning may be achieved by washing or rinsing to remove any remaining substrate or debris. After washing, depending on the intended use of the larvae, they may need to be dried to reduce their moisture content and increase their shelf life. This can be done using heat or air-drying methods. Lastly, the larvae are then packaged in appropriate containers, such as bags or boxes, for transportation or storage.

In this process, the step of separating the desired larvae from the substrate by manual or mechanical means is slow and labor intensive, which result in increased costs of production. A solution is needed that accelerates the larvae-substrate separation processing time and that reduces cost of production. Such as time and production cost reduction solution are provided below.

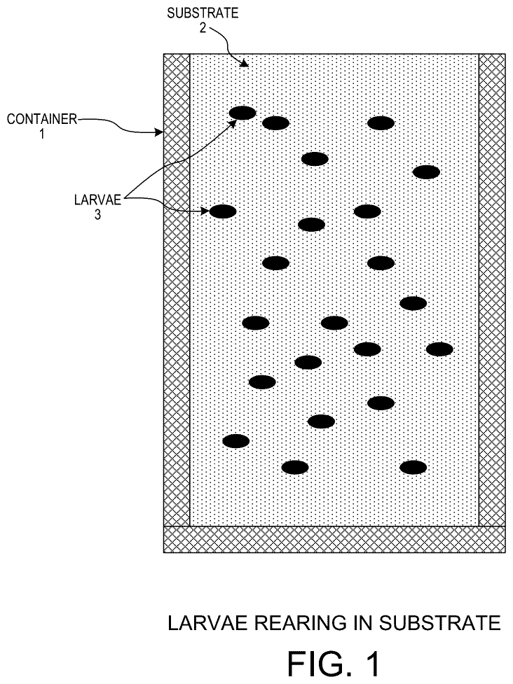

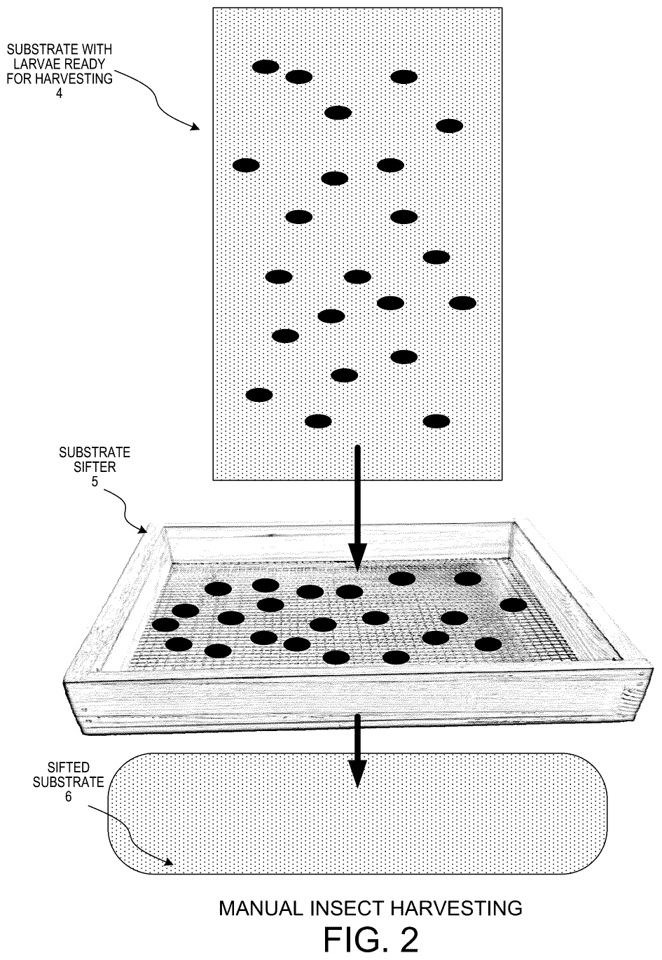

FIG. 1 is a first diagram of a container housing insects being reared in a biological substrate. In one embodiment, the insects being rear are Black Soldier Fly Larvae (BSFL) and the substrate includes coffee bean biological material. The container 1 is partially filled with the coffee been substrate 2. The insect larvae 3 are then placed on the substrate 3 within the container 1. After a period of time, the insect larvae consume at least a portion of the substrate 2. After consuming the substrate 2, the insect larvae 3 grow in size and weight. After the insect larvae have grow substantially, but have not yet completed their larvae maturation process, it is desirable to harvest the insect larvae from the container 1. In order to harvest the insect larvae, the insect larvae need to be separated from the substrate 2. Separating the insect larvae from the substrate is time consuming and labor intensive. An example of time consuming and labor intensive insect larvae separation is illustrated in FIG. 2.

FIG. 2 is a diagram of manual insect harvesting utilizing a sifter. Once the insect larvae in the substrate are ready for harvesting, the insect larvae must be separated from the substrate. This can and is often achieved by way of manual use of a substrate sifter 5. The substrate shifter 5 includes a perimeter wall to prevent material from flowing over the edges of the substrate shifter 5. Between the perimeter wall a wire or plastic mesh is connected.

In operation, the substrate including the desired insect larvae are poured onto a substrate shifter 5. Then, by combination of gravity and horizontal “sifting” force, the substrate passes through the mesh. However, the mesh is sized such that the desired insect larvae do not pass through the mesh and therefore remain resting upon the mesh after all the substrate has passed through the mesh to the pile of sifted substrate 6. The insect larvae that remain resting on the mesh are then moved to an insect larvae collection bin for packaging or further processing.

It is readily apparent that this manual method of harvesting insect larvae from a substrate is slow and labor intensive. Moreover, this manual method can cause loss of some smaller insect larvae that may pass through the mesh. It is also well known that all manual processes suffer from the inconsistent operation by human operators. Accordingly, a faster and less labor intensive technology is needed.

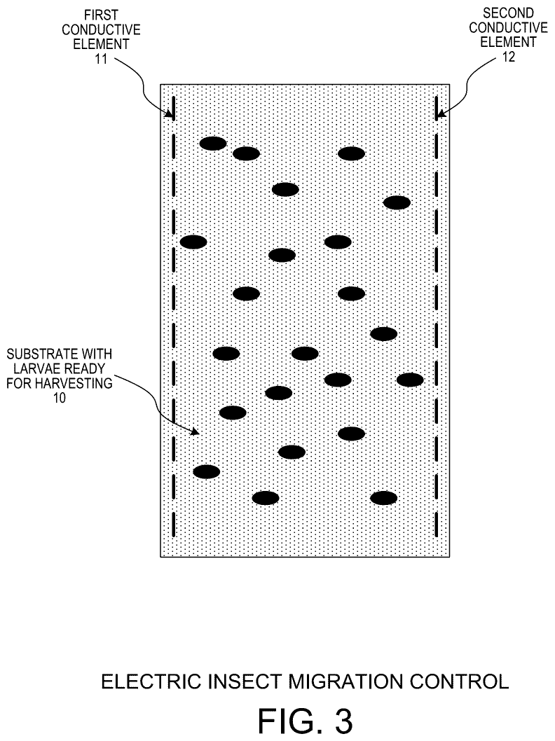

FIG. 3 is a first simplified diagram of an electric insect migration control system. After a great deal of experimentation, the electric insect migration control system illustrated in FIG. 3 was created. The electric insect migration control system in its simplest form includes a container including a first conductive element 11 and a second conductive element 12. The first and second conductive elements 11, 12 are positioned such that the majority of the substrate with larvae ready for harvesting 10 are located between first conductive element 11 and second conductive element 12.

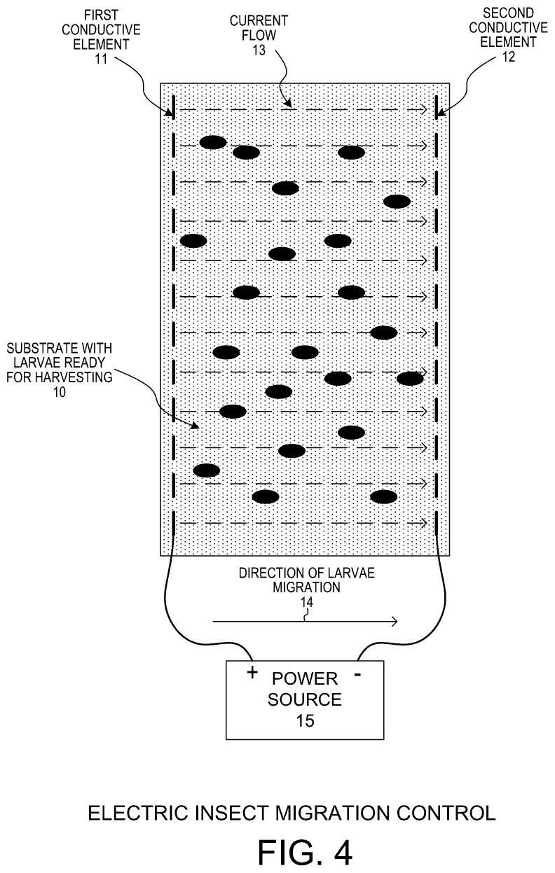

FIG. 4 is a second simplified diagram of an electric insect migration control system connected to a power source. The power source 15 is configured to apply electrical potential across the first and second conductive elements 11, 12. In one embodiment, the positive terminal of power source 15 outputs thirty (30) volts and the negative terminal of power source 15 outputs zero (0) volts.

In operation, when the power source 15 positive output is electrically connected to the first conductive element 11 and the power source 15 negative output is electrically connected to the second conductive element 12, a current 13 is generated and flows between the first conductive element 11 and the second conductive element 12. The current 13 flows through both the substrate 10 as well as the insect larvae being reared in the substrate.

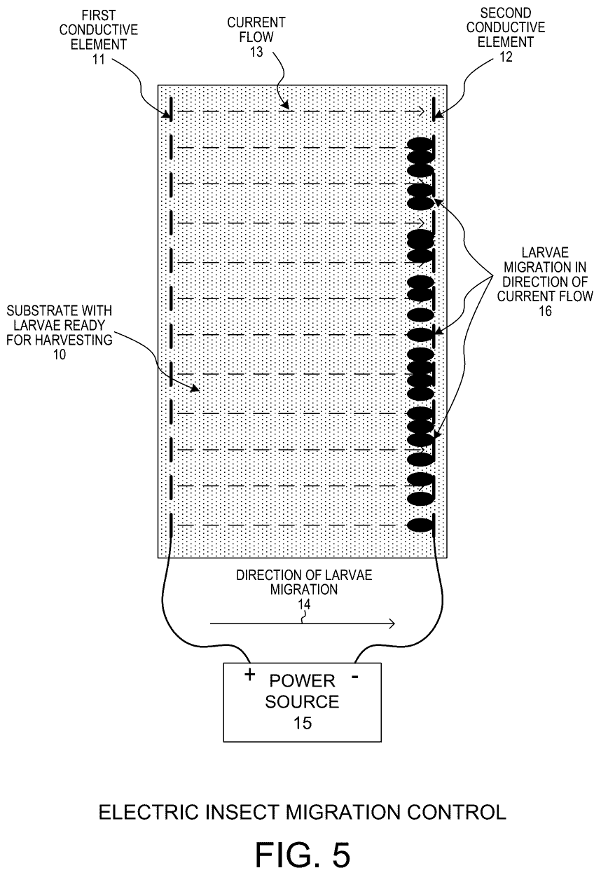

A very interesting phenomenon has been discovered by the Applicant in this scenario. When the electrical current is applied to the insect larvae in the substrate, the insect larvae respond by moving in the direction of the current flow. For example, referring back to FIG. 4, when the electric potential is applied to the first and second conductive elements 11, 12 and resulting current 13 flows through the insect larvae in the substrate, the insect larvae respond by starting a migration toward the second conductive element 12. This is referred to as the direction of larvae migration 14. The result of this migration is illustrated in FIG. 5.

FIG. 5 is a third simplified diagram of an electric insect migration control system connected to a power source after the insect migration is completed. As is illustrated, all of the insect larvae have migrated toward the second conductive element 12. This discovered phenomenon is not only very interesting, but it can also be applied in a very practical manner. One very practical manner of application is described herein below.

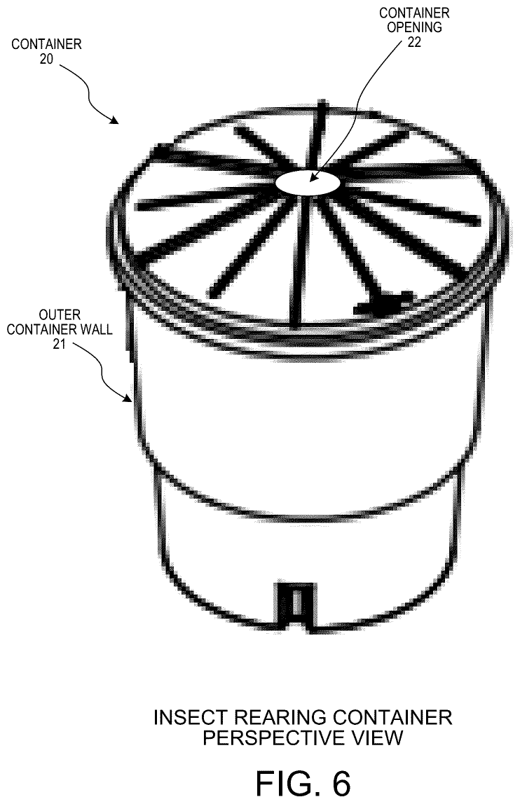

FIG. 6 is a perspective view diagram of an insect rearing container. The insect rearing container 20 has an outer wall 21 that functions to hold substrate material within the container. The insect rearing container 20 also includes a contain lid with an opening 22. The opening functions to provide access to the interior of the container 20. The insect rearing container 20 may be utilized to house an electric insect migration harvesting apparatus as described below.

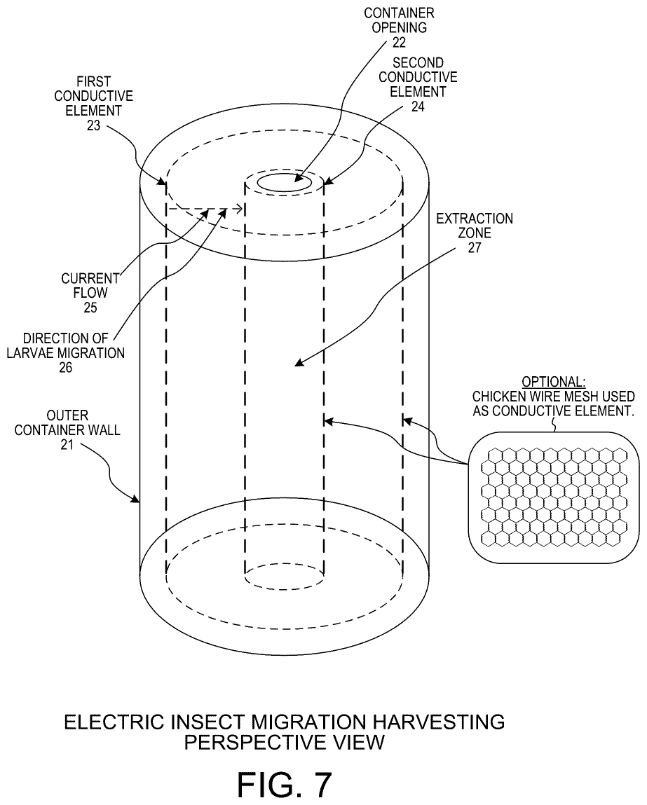

FIG. 7 is a perspective view diagram of the internal parts of an electric insect migration harvesting apparatus. The electric insect migration harvesting apparatus includes a first conductive element 23, a second conductive element 24, an opening 22, an outer wall 21 and extraction zone 27. Given the cylindrical shape of the container, the first conductive element and the second conducive element are also constructed in a cylindrical shape. The first conductive element and the second conductive element can be constructed using any electrically conductive material. In one embodiment, the first conductive element and the second conductive element are constructed with chicken wire mesh. The chicken wire mesh not only provides the function of electrically conductive element, but it also provides a function of separating the substrate material from the extraction zone. Maintaining open space in the extraction zone greatly aids the insect larvae harvesting process described herein below.

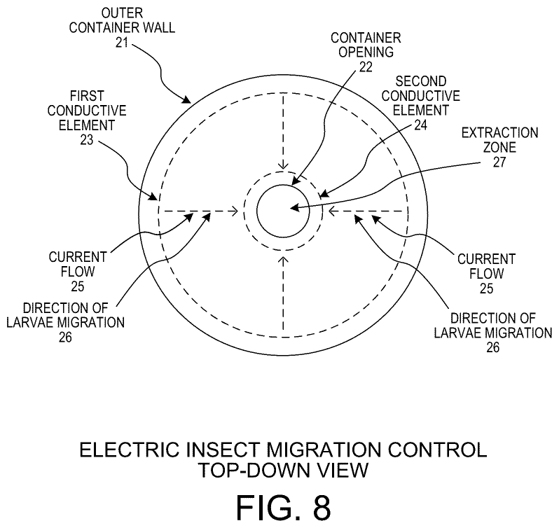

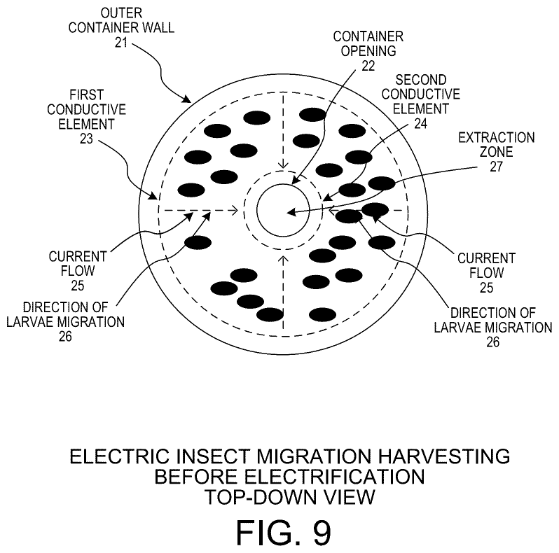

FIG. 8 is a top-down view diagram of the internal parts of an electric insect migration harvesting apparatus. As discussed above, the electric insect migration harvesting apparatus includes a first conductive element 23, a second conductive element 24, an opening 22, an outer wall 21 and extraction zone 27. An operational example illustrating the migration of insect larvae is provided in FIG. 9.

FIG. 9 is a top-down view diagram of the internal parts of an electric insect migration harvesting apparatus housing a plurality of insect larvae. In a first instance, the insect larvae are in the rearing stage feeding on the substrate biological material and growing in size. During this stage no electrical potential is applied to either the first or second conductive elements 23, 24. Accordingly, there is no current flow 25 between the first conductive element 23 and the second conductive element 24.

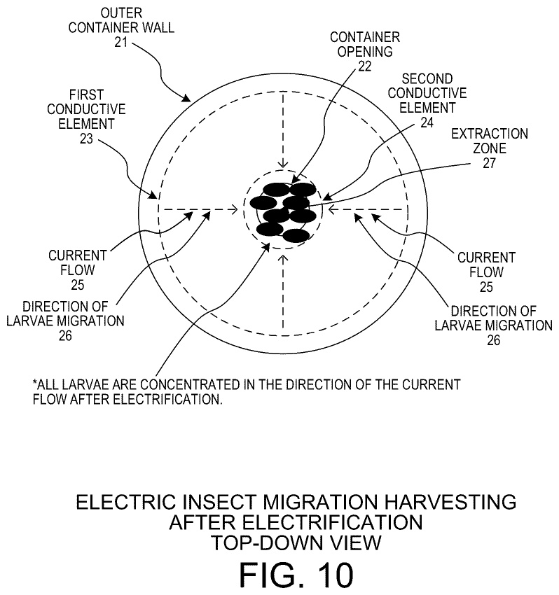

In a second instance, the insect larvae have completed the rearing stage and are ready for harvesting from the substrate. During this stage the electrical potential is applied to the first conductive element 23 and the second conductive element 24. Accordingly, current 25 begins to flow between the first conductive element 23 and the second conductive element 24. Current flow 25 in turn causes the insect larvae to start migrating toward the second conductive element 24. The result of the migration is illustrated in FIG. 10.

FIG. 10 is a top-down view diagram of the internal parts of an electric insect migration harvesting apparatus after a plurality of insect larvae have migrated towards the second conductive element. During the migration, the insect larvae migrate toward the second conductive element 24. When the insect larvae arrive at the second conductive element 24, which is constructed with chicken wire mesh, the insect larvae fall pass through the chicken wire mesh and fall into the extraction zone 27. As discussed above, the chicken wire mesh functions not only as a conductive element, but it also functions as a barrier that separates the substrate material and the open space in the extraction zone. Therefore, once the insect larvae pass through the chicken wire mesh of the second conductive element 24, the insect larvae fall into the open space of the extraction zone 27.

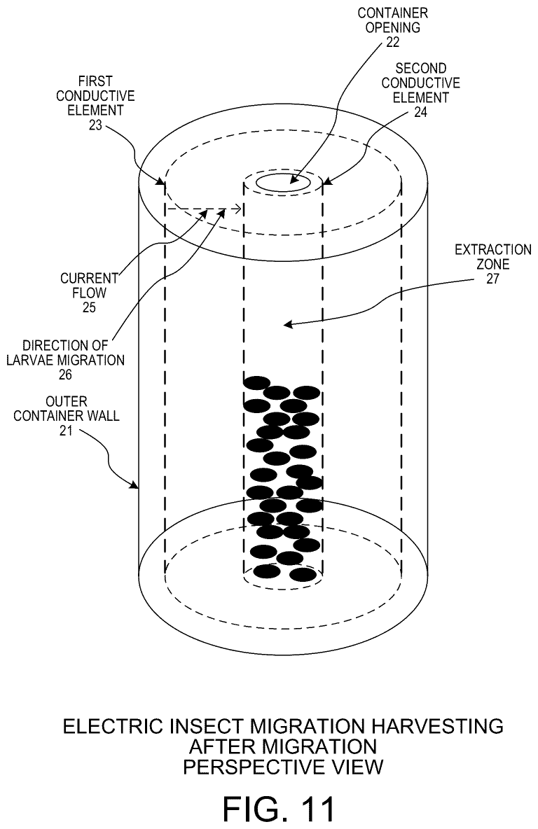

FIG. 11 is a perspective view diagram of the internal parts of an electric insect migration harvesting apparatus after insect migration has been completed. This perspective view of the electric insect migration harvesting apparatus after insect migration has been completed, illustrates how the migrated insect larvae are conveniently gathered into the extraction zone free from the substrate. The insect larvae now located in the extraction zone 27 are ready for extraction.

In one example, the insect larvae can be extracted by rotating the container and pouring the insect larvae out of the container opening 22.

In another example, the insect larvae can be extracted by way of a vacuum force. Application of a vacuum force to the extraction zone via the opening 22 will cause the migrated insect larvae to be vacuumed out of the extraction zone and into a desired location outside of the container. Application of a vacuum force on the extraction zone may be performed in different manners.

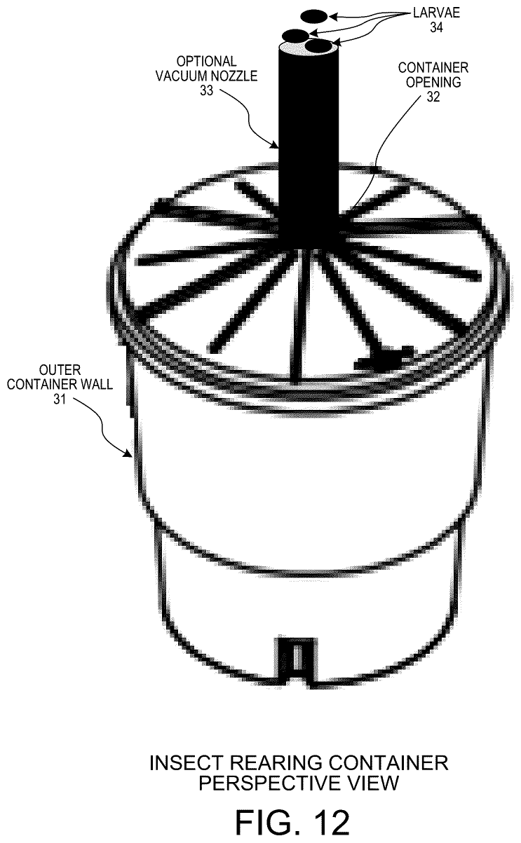

FIG. 12 is a perspective view diagram of an insect rearing container including an optional vacuum nozzle. FIG. 12 illustrates a first manner of applying a vacuum force on the extraction zone. An optional vacuum nozzle 33 may be inserted into the opening 32 of the container. Once the optional vacuum nozzle 33 is inserted, the vacuum force can be applied to create the necessary suction to vacuum the migrated insect larvae out of the extraction zone.

In another embodiment, the optional vacuum nozzle may be permanently attached to the container. This embodiment would allow for a vacuum hose to be attached to quickly to a container without the need for mounting the nozzle.

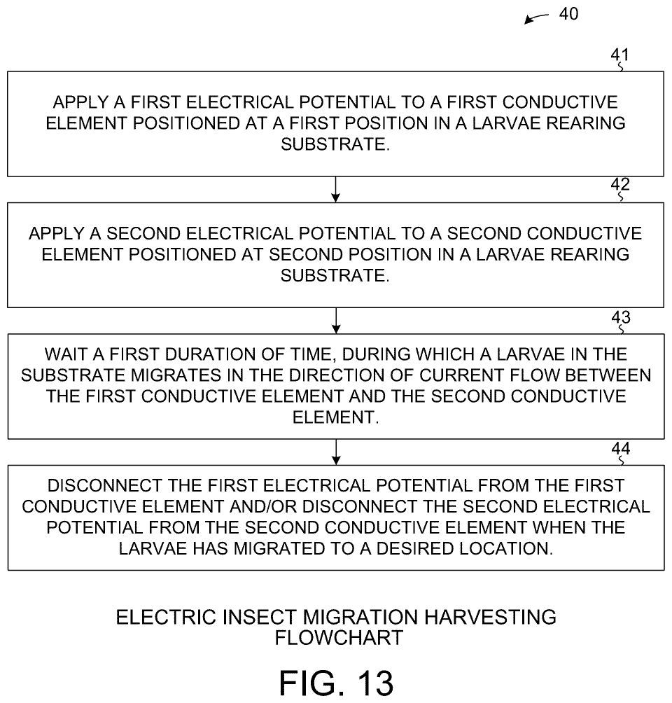

FIG. 13 is a flowchart diagram 40 illustrating the steps of electric insect migration harvesting. In step 41, a first electrical potential is applied to a first conductive element positioned at a first position in the insect larvae rearing substrate. In step 42, a second electrical potential is applied to a second conductive element positioned at a second position in a larvae rearing substrate. In step 43, a first duration of time passes during which an insect larvae in the substrate migrates in the direction of the current flow between the first conductive element and the second conductive element. In step 44, the first electrical potential is disconnected from the first conductive element and/or the second electrical potential is disconnected from the second conductive element when the larvae has migrated to a desired location.

It is noted herein, that while the above embodiments utilize a cylindrical shape, other shapes are useable and fall within the scope of this disclosure and claims. For example, the shape me be rectangular wherein the first conductive element and the second conductive element only extend along a two-dimensional plane. The exact shape of the conductive elements may vary, however, so long as a current flow is generated between the two conductive elements the insect larvae located there in between will begin to migrate in the direction of the current flow.

It is noted herein that the container may be made of a variety of different material and be made in a variety of different shapes. The exemplary container illustrated herein, is only one embodiment illustrating how the present invention could be practiced.

Although certain specific embodiments are described above for instructional purposes, the teachings of this patent document have general applicability and are not limited to the specific embodiments described above. Accordingly, various modifications, adaptations, and combinations of various features of the described embodiments can be practiced without departing from the scope of the invention as set forth in the claims.

What is claimed is:

1. An apparatus, comprising:
a container;
a first conductive element;
a second conductive element, wherein the first conductive element and the second conductive element are positioned within the container;
an extraction zone, wherein the extraction zone is located within the container;
an access configured to provide access to the extraction zone from outside of the container;
a positive electric terminal; and
a negative electric terminal, wherein the positive electric terminal is electrically connected to the first conductive element, and wherein the negative electric terminal is electrically connected to the second conductive element, and wherein the second conductive element is located closer to the extraction zone than the first conductive element.

2. The apparatus of claim 1, further comprising:
an electric energy source, wherein a positive terminal of the electric energy source is electrically connected to the positive electric terminal, and wherein a negative terminal of the electric energy source is electrically connected to the negative electric terminal.

3. The apparatus of claim 1, further comprising:
a switch configured to connect or disconnect a terminal of the electric energy source to the first conductive element or the second conductive element.

4. The apparatus of claim 1, wherein the container is configured to house a biological substrate material.

5. The apparatus of claim 4, wherein the first conductive element and the second conductive element are positioned within the container such that the majority of an interior volume of the container is between the first conductive element and the second conductive element.

6. The apparatus of claim 1, wherein the first conductive element and the second conductive element are configured such that a current flows between the first conductive element and the second conductive element when a first electric potential is applied to the first conductive element and a second electric potential is applied to the second conductive element.

7. The apparatus of claim 1, wherein the container is configured to rear an insect.

8. The apparatus of claim 1, wherein the container is configured to be heated or cooled.

9. The apparatus of claim 1, wherein the container is configured to allow entry of air from outside the container.

10. The apparatus of claim 1, wherein the container is configured to allow entry of water from outside the container.

11. The apparatus of claim 1, further comprising a vacuum tube configured to apply a vacuum to the extraction area.

12. The apparatus of claim 11, wherein the vacuum tube comprises a substrate filter configured to block the flow of substrate while allowing the passage of a larvae.

13. The apparatus of claim 1, wherein the access is configured to mate with a vacuum nozzle.

14. The apparatus of claim 13, further comprising a lid configured to cover the access.

15. A method, comprising:
(a) applying a first electrical potential to a first conductive element positioned at a first position in a biological substrate;
(b) apply a second electrical potential to a second conductive element position at a second position in the biological substrate;
(c) waiting a first duration of time, wherein an insect in the biological substrate moves in a direction of current flow between the first conductive element and the second conductive element; and
(d) disconnecting the first electrical potential from the first conductive element or disconnecting the second electrical potential form the second conductive element.

16. The method of claim 16, further comprising:
(e) collecting the insect from an extraction zone located adjacent to the second conductive element.

17. An apparatus, comprising:
a container;
an extraction zone, wherein the extraction zone is located within the container;
a positive electric terminal;
a negative electric terminal; and
a first means for causing one or more insects in the container to move in a first direction.

18. The apparatus of claim 19, wherein the first means comprises:
a first conductive element; and
a second conductive element, wherein the positive electric terminal is electrically connected to the first conductive element, and wherein the negative electric terminal is electrically connected to the second conductive element.

19. The apparatus of claim 17, further comprising:
a second means for extracting the one or more insects from the extraction zone.

20. The apparatus of claim 19, wherein the second means comprises:
an opening that provides access to the interior of container.
